SEQUENCE LISTING (14 sequences)

SEQ ID NO: 1 - DNA, 20 bp, Artificial Sequence
  Description of Artificial Sequence Synthetic primer, Locus a
ttcccactcaaaactcccac

SEQ ID NO: 2 - DNA, 20 bp, Artificial Sequence
  Description of Artificial Sequence Synthetic primer, Locus a
acaggaatgagggcagctaa

SEQ ID NO: 3 - DNA, 21 bp, Artificial Sequence
  Description of Artificial Sequence Synthetic primer, Locus b
tgcccgcgtccgtccgtgaaa

SEQ ID NO: 4 - DNA, 22 bp, Artificial Sequence
  Description of Artificial Sequence Synthetic primer, Locus b
agtctccgtcgccgtcctcgtc

SEQ ID NO: 5 - DNA, 23 bp, Artificial Sequence
  Description of Artificial Sequence Synthetic primer, Locus c
ggtaggatgattctagaatgaca

SEQ ID NO: 6 - DNA, 20 bp, Artificial Sequence
  Description of Artificial Sequence Synthetic primer, Locus c
gcccaaattggcttcttttt

SEQ ID NO: 7 - DNA, 22 bp, Artificial Sequence
  Description of Artificial Sequence Synthetic primer, Locus d
gtctttagctgctgaggaaatg

SEQ ID NO: 8 - DNA, 21 bp, Artificial Sequence
  Description of Artificial Sequence Synthetic primer, Locus d
agcagaattctgcacatgacg

SEQ ID NO: 9 - DNA, 19 bp, Artificial Sequence
  Description of Artificial Sequence Synthetic primer, Locus e
caactggccctgtcgttcc

SEQ ID NO: 10 - DNA, 20 bp, Artificial Sequence
  Description of Artificial Sequence Synthetic primer, Locus e
ccatgttgctgacccggtag

SEQ ID NO: 11 - DNA, 22 bp, Artificial Sequence
  Description of Artificial Sequence Synthetic primer, Locus f
actggttggagttgtggagacg

SEQ ID NO: 12 - DNA, 21 bp, Artificial Sequence
  Description of Artificial Sequence Synthetic primer, Locus f
tggaatgcttgaaggctgctc

SEQ ID NO: 13 - DNA, 21 bp, Artificial Sequence
  Description of Artificial Sequence Synthetic primer, Locus g
aactgaatggcagtgaaaaca

SEQ ID NO: 14 - DNA, 19 bp, Artificial Sequence
  Description of Artificial Sequence Synthetic primer, Locus g
ccctagcctgtcattgctg

(19) United States
(12) Patent Application Publication
(10) Pub. No.: US 2013/0065776 A1
(43) Pub. Date: Mar. 14, 2013

(54) SELECTIVE ENRICHMENT OF NON-METHYLATED NUCLEIC ACIDS

(71) Applicants: Chrìstìan Korfhage, Langenfeld (DE); Andreas Meier, Duesseldorf (DE)

(72) Inventors: Chrìstìan Korfhage, Langenfeld (DE); Andreas Meier, Duesseldorf (DE)

(73) Assignee: QIAGEN GMBH

(21) Appl. No.: 13/512,144

(22) PCT Filed: Nov. 23, 2010
(86) PCT No.: PCT/EP10/68006

§ 371 Date: Nov. 12, 2012

(30) Foreign Application Priority Data
Dec. 4, 2009 (DE) 10 2009 057 702.5

(51) Int. Cl. C12Q 1/68; C40B 30/04; C12P 19/34

(52) U.S. Cl. 506/9; 435/91.2; 435/6.12; 435/6.11

(57) ABSTRACT

The present invention relates to a method for selectively amplifying non-methylated sequences of a DNA comprising the steps of (i) providing a sample comprising a DNA which is methylated at least one site, (ii) treating the DNA in the sample with a methylation-dependent nuclease, and (iii) amplifying the DNA cut using the methylation-dependent nuclease. In addition, the invention relates to kits for use in the method according to the invention. The method according to the invention can be used for selectively preparing to (selectively accumulating) non-methylated sequence segments of genomic DNA and for analysing the global methylation pattern in genomic DNA.

### TECHNICAL FIELD OF THE INVENTION

The present invention is in the field of biology and chemistry, more particularly molecular biology. Specifically, the invention relates to the amplification of non-methylated regions of nucleic acids and to the analysis of methylation patterns in nucleic acids.

### BACKGROUND OF THE INVENTION

Methylation is a commonly occurring chemical modification of DNA, in which methyl groups have been transferred to nucleobases, for example at the carbon-5 position of the cytosine pyrimidine ring. This generally occurs by means of specific DNA methyltransferases, either de novo or to maintain an existing methylation pattern, for instance during DNA replication.

DNA methylation can have multiple functions: for example, it can be used by prokaryotes to distinguish endogenous DNA from foreign DNA introduced into the prokaryote. In addition, it has among other things an important role in error correction during DNA synthesis in prokaryotes, allowing the original (template) strand to be distinguished from the newly synthesized strand. Many prokaryotes have DNA methyltransferases which methylate endogenous DNA at or in the proximity of particular signal sequences. In these organisms, foreign, unmethylated DNA can be cut at or in the proximity of signal sequences by specific methylation-sensitive restriction endonucleases and thus degraded.

Besides the aforementioned methylation-sensitive endonucleases which only cut non-methylated regions, there are also methylation-dependent endonucleases which only cut at or next to particular methylated sequences.

In eukaryotes, the methylation of DNA provides an additional layer of information, for example allowing active regions of the genome to be distinguished from inactive regions. Methylation patterns have a particular role especially in differential gene expression and are therefore also relevant in the development of tumours.

To obtain information regarding methylation patterns in DNA, a range of methods from the prior art are known to a person skilled in the art: in bisulphite sequencing for example, the DNA to be analysed is first reacted with bisulphite so that the non-methylated cytosines are converted into uracil, followed by amplification by means of PCR and by DNA sequencing. From the sequence differences between bisulphite-treated and non-bisulphite-treated DNA, the underlying methylation pattern can be inferred. Alternatively, it is also possible to use methylation-specific PCR (MSP) to analyse the bisulphite-treated DNA, using methylation-specific primers, i.e. primers which are complementary to the unconverted sequence. As an alternative to the bisulphite technique, it is possible to use other methods, such as methylation-specific restriction analysis or methylated DNA immunoprecipitation (MeDIP).

The aim of these methods is the analysis of the methylation of defined sequence regions and the quantification of the degree of methylation of defined sequence regions.

### DESCRIPTION OF THE INVENTION

The present invention provides a method with which the global methylation pattern of a DNA can be established. Thus, the aim of the method according to the invention is primarily the identification of genomic segments containing methylated regions. In particularly preferred embodiments, the aim of the invention is the identification of genomic segments containing methylated regions and not the determination of methylation states of particular individual bases.

Using the method according to the invention, it is also possible to perform selective preparation of non-methylated DNA.

The method consists of several sub-steps:
(1) DNA, preferably genomic DNA, is digested using a methylation-dependent nuclease.
(2) After digestion, the DNA is duplicated by means of an amplification method, preferably a random-primed sequence amplification method, so only the non-methylated DNA segments—i.e. those segments which were not cut—can be duplicated. The result of the amplification is the duplicated DNA without those segments which were cut earlier by the methylation-dependent nuclease. Accordingly, the method results in selective accumulation and duplication of sequence sections which are not methylated.
(3) Optionally, it is subsequently possible to carry out quantitative analysis of the copy number of sequence segments in the DNA selected and amplified according to the method.

The latter can be used to analyse the methylation pattern of a DNA or sub-segments thereof, i.e. to what extent a DNA or a defined part thereof was originally methylated.

The method of the present invention is therefore complementary to other methods for analysing the methylation of nucleic acids.

The method according to the invention can aid the identification of genomic segments which are methylated. By amplifying the selected sequences, the method permits global methylation analysis without having to know the exact methylation site.

Thus, the invention provides a method for selectively amplifying non-methylated sequences of a DNA comprising the steps of
(i) providing a sample comprising a DNA which is methylated at least one site,
(ii) treating the DNA in the sample with a methylation-dependent nuclease, and
(iii) amplifying the DNA cut using the methylation-dependent nuclease.

Steps (ii) and (iii) can be carried out at the same time (simultaneously) or in succession.

A nuclease is an enzyme which hydrolytically cleaves a nucleic acid (e.g. genomic DNA). In this process, the phosphodiester bonds are hydrolytically cleaved. Preferred in the context of the invention are endonucleases. A nuclease is methylation-dependent when the enzyme can only bind to methylated sites or can only cleave methylated sites. Examples of such methylation-dependent nucleases include the enzymes McrBC, McrA and MrrA. McrA cuts m5CG-methylated DNA, McrBC cuts (A/G)m5C-methylated DNA, and MrrA cuts m6N adenine-methylated DNA. The methylation-dependent nuclease is preferably selected from the group consisting of McrBC, McrA, DpnI, BisI, BlsI, GlaI, GluI, MalI and PcsI. Such nucleases are described, for example, in Chmuzh et al. (2005) (BMC Microbiology 6: 40i), Tarasova et al. (2008) (BMC Molecular Biology 9: 7), Chemukhin et al. (2007a) (Ovchinnikov bulletin of biotechnology and physical and chemical biology V.3, No. 1, pp. 28-33) and Chemukhin et al. (2007b) (Ovchinnikov bulletin of biotechnology and physical and chemical biology V.3, No. 2, pp. 13-17). Preferred methylation-dependent nucleases in the context of the present invention are McrBC and McrA, and particular preference is given to McrBC. McrBC is commercially available, for example from New England Biolabs Inc., Ipswich, Mass., USA. In addition, it is also possible to use homologues of the aforementioned nucleases, for example the McrBC-homologous enzyme systems described in Fukuda (2008) (Genome Biol. 9(11): R163). LlaJI has also been described as an McrBC homologue (O'Driscoll (2006), BMC Microbiology 2006, 6: 40i). Also suitable in particular embodiments of the present invention are nucleases whose specificity has been altered in such a way, for example by use of new buffer conditions, modification(s), amino acid substitution(s) or other manipulations, that they can cut semi-methylated or completely methylated regions. Such enzymes are described, for example, in Formenkov et al. (2008) (Anal. Biochemistry 381: 135-141).

Alternatively, a methylated base can be excised from the DNA by a DNA glycosylase. The result is that this site cannot be amplified in a subsequent amplification reaction. For example, 5-methylcytosine can be excised by a 5-methylcytosine DNA glycosylase. The efficiency of the amplification stop at the abasic site can be supported by a corresponding lyase which cuts the sugar-phosphate DNA backbone at the abasic site. The method can also result here in a strand break, for example by the use of enzymes (e.g. lyases) or appropriate reaction conditions.

All aforementioned enzymes and enzyme systems can therefore, under suitable conditions, be considered to be methylation-dependent nucleases in the broader sense, provided they exhibit the appropriate activity.

The amplification can in principle be carried out by means of isothermal or non-isothermal methods. Examples of known isothermal amplification methods are strand displacement amplification (SDA), multiple displacement amplification (MDA), rolling circle amplification (RCA), loop-mediated isothermal amplification (LAMP), transcription-mediated amplification (TMA), helicase-dependent amplification (HDA), SMart amplification process (SMAP), single primer isothermal amplification (SPIA). Examples of known non-isothermal amplification methods are the ligase chain reaction (LCR) and the polymerase chain reaction (PCR). Preferred in the context of the present invention are random-primed sequence amplification methods. These can be isothermal or non-isothermal. Examples of non-isothermal random-primed sequence amplification methods are random-primed PCR methods such as PEP-PCR (primer extension preamplification PCR), iPEP-PCR (improved primer extension preamplification PCR), DOP-PCR (degenerate oligonucleotide primer PCR), adaptor-ligation PCR or methods such as OmniPlex® (Sigma-Aldrich) or GenomePlex® (Rubicon). Examples of preferred isothermal sequence amplification methods are strand displacement reactions which include, for example, strand displacement amplification (SDA) in the narrower sense and multiple displacement amplification (MDA), rolling circle amplification (RCA), single primer isothermal amplification (SPIA) and all subtypes of these reactions, such as restriction-aided RCA (RCA-RCA), MDA with nested primers, linear and exponential strand displacement reactions and helicase-dependent amplification (HDA). Particularly preferred examples of isothermal random-primed sequence amplification methods in the context of the present invention are MDA and RCA. All these methods are known to a person skilled in the art (cf., for example, US 2005/0112639 A1, US 2005/0074804 A1, US 2005/0069939 A1 and US 2005/0069938 A1, and Wang G. et al. (2004), Genome Res. November; 14(11): 2357-2366; Milla M. A. et al. (1998), Biotechniques March; 24(3): 392-396; Nagamine K. et al. (2001), Clin Chem. 47(9): 1742-1743; Lage J. M. et al. (2003), Genome Res. 13(2): 294-307 and Vincent M. et al. (2004), EMBO Rep. 5(8): 795-800). A strand displacement reaction is understood here to mean all reactions in which a polymerase is used which exhibits strand displacement activity.

Strand displacement activity of a polymerase means that the enzyme used is capable of separating a nucleic acid double strand into two individual strands. Examples of DNA polymerases having strand displacement activity which, for example, can be used in RCA are holoenzymes or parts of replicases from viruses, prokaryotes, eukaryotes, or archaea, Phi 29-type DNA polymerases, the DNA polymerase Klenow exo- and the DNA polymerase from Bacillus stearothermophilus having the designation Bst exo-. “exo-” means that the corresponding enzyme does not exhibit any 5′-3′ exonuclease activity. A known representative of the Phi 29-type DNA polymerases is the DNA polymerase from the bacteriophage Phi 29. Other Phi 29-type DNA polymerases occur, for example, in the phages Cp-1, PRD-1, Phi 15, Phi 21, PZE, PZA, Nf, M2Y, B103, SF5, GA-1, Cp-5, Cp-7, PR4, PRS, PR722 and L 17. Further suitable DNA polymerases having strand displacement activity are known to a person skilled in the art. Alternatively, DNA polymerases having strand displacement activity are also understood to mean DNA polymerases without strand displacement activity if, in addition to an appropriate DNA polymerase, use is made of a catalyst, for example a protein or a ribozyme, which allows the separation of a DNA double strand or the stabilization of individual DNA strands. These proteins include, for example, the helicases, SSB proteins and recombination proteins which may be present as constituent of larger enzyme complexes such as replicases for example. In this case, using components in addition to the polymerase, a polymerase having strand displacement activity is generated. The polymerases having strand displacement activity can be heat-labile or heat-stable.

In one particular embodiment, the polymerase used for the amplification and having strand displacement activity is a Phi 29-like polymerase, preferably a polymerase from a phage selected from a group of phages comprising Phi 29, Cp-1, PRD-1, Phi 15, Phi 21, PZE, PZA, Nf, M2Y, B103, SF5, GA-1, Cp-5, Cp-7, PR4, PR5, PR722 and L 17. Particular preference is given to the use of the polymerase from the phage Phi 29.

It is apparent to a person skilled in the art that the use of mixtures of two or more polymerases having strand displacement activity is also possible. Furthermore, it is also possible for one or more polymerases having strand displacement activity to be combined with one or more polymerases without strand displacement activity.

In the context of the present invention, MDA and RCA are preferred amplification methods. Preference is also given to carrying out an amplification of the whole genome (whole genome amplification (WGA)). WGA means that the template DNA is, in principle, to be substantially completely amplified, though according to the invention, only the non-methylated part of the genome is to be amplified.

Therefore, in preferred embodiments, the invention provides the amplification of genomic DNA.

According to the invention, the DNA amplification is preferably carried out in a random-primed sequence amplification method (RPSA), i.e. the priming of the amplification reactions is done randomly, for example via primers having a randomly chosen sequence (random primers). Against the background of the present invention, a random-primed sequence amplification method is understood to mean the amplification of genomic DNA wherein the primers used bind in a random manner to the DNA, preferably genomic DNA. The randomness of the binding of primers to the DNA, preferably genomic DNA, can be established by different means: it is possible to use random primers for the amplification. Random primers have the sequence NNNNNN for a hexamer primer for example, where N is any desired nucleotide. As a result, random primers can contain all possible sequences. Alternatively, it is also possible to use primers having degenerate sequences. These primers can include, for example, particular sequence motifs, with random sequences being interspersed at some positions of the primer.

It is also possible to use primers having a particular sequence, though it must be ensured that these primers bind with sufficient frequency to the target DNA. This can, for example, be ensured by said primers being short or the primer binding conditions being adjusted such that unspecific binding is allowed.

In an RPSA, the majority of a genomic nucleic acid is amplified. If a plurality of different genomic nucleic acids is present as template nucleic acid, the reaction conditions can be chosen such that all genomic nucleic acids, only one genomic nucleic acid, but at least a complex part of the genomic nucleic acid of the template nucleic acid is amplified. The complexity of the amplified part of the genomic nucleic acid is between 10 000 and 100 000 nt, particularly preferably 100 000 to 1 000 000, and in another particularly preferred embodiment greater than 1 000 000 nt. Examples of RPSA methods are multiple displacement amplification (MDA), rolling circle amplification (RCA), random-primed PCR techniques such as degenerate oligonucleotide primer PCR (DOP-PCR) and primer extension preamplification PCR (PEP-PCR). Other suitable PCR methods attach primer binding sites to, for example, DNA fragments which, for example, are formed as a result of cutting the DNA using restriction endonucleases or as a result of ultrasonication. Further suitable PCR methods use, in a first step, primers which bring about random primer binding by means of their 3′ end, but introduce a specific primer binding site using their 5′ end. Subsequently, PCR takes place with the primers which hybridize to specific primer binding site. This principle can also be carried out in an RPSA according to the invention.

In one particular embodiment of the invention, therefore, after treatment of the DNA in a sample, an RPSA is carried out to amplify the DNA cut at the methylation sites. In this embodiment, preferably no ligation reaction is carried out after the restriction using the methylation-dependent nuclease and before the RPSA.

Polymerases are enzymes which catalyse the formation of phosphodiester bonds between individual nucleotides within a nucleic acid strand (e.g. DNA and RNA polymerases). Both heat-labile polymerases and non-heat-labile polymerases can be used. Particular preference is given to all heat-labile and non-heat-labile polymerases which exhibit strand displacement activity under the chosen experimental conditions. Appropriate polymerases are commercially available and known to a person skilled in the art.

Amplification of a nucleic acid is understood to mean the multiplication of the template by at least a factor of 2 or more. For this purpose, the nucleic acid can be multiplied linearly or exponentially. Linear amplification can be achieved, for example, by means of RCA in the presence of primers which hybridize on the target circle to only one specific sequence. Exponential amplification can be achieved, for example, via RCA with primers wherein the primers hybridize to at least 2 binding sites on the target circle or else hybridize to at least one binding site on the target circle and at least one binding site on the complementary strand. A person skilled in the art is familiar with further linear and exponential amplification methods suitable for the present invention, for example MDA or PCR.

According to the invention, an isothermal reaction is understood to mean a reaction which is carried out at only one temperature. If the reaction is brought to another temperature before the start (e.g. on ice) or at the end of the reaction (e.g. in order to inactivate reaction components or enzymes), the reaction is still termed isothermal provided the actual reaction is carried out at a constant temperature. A temperature is understood to be constant when the temperature fluctuation does not exceed +/−10° C., preferably +/−5° C.

In the context of the present invention, a primer is understood to mean a molecule which is used as a start site for an enzyme having nucleic acid polymerase activity. Said primer can be a protein, a nucleic acid or another molecule which a person skilled in the art finds to be suitable as polymerase start site. Said molecule can be used as a start site via an intermolecular interaction and also via an intramolecular interaction. In the case of nucleic acid primers, they do not have to, but can hybridize across their entire length to the template nucleic acid. Preference is given to nucleic acid primers, more particularly oligonucleotides.

Particular preference is given to the use of random primers for the amplification of the DNA, i.e. a primer mixture comprising a plurality of different primers of random sequence.

Besides random primers, other primers can also be used for the amplification of the nucleic acid(s). As mentioned, degenerate and/or sequence-specific primers can also be used for the amplification of the DNA.

The primers used for the amplification typically comprise 4 to 35 nucleotides, preferably between 5 to 25 nucleotides, particularly preferably 6 to 15 nucleotides.

In one preferred embodiment, the method according to the invention comprises the additional step of
(iv) detecting at least one sequence segment of the amplified DNA.

The detection preferably comprises the quantification of at least one sequence segment (locus) of the amplified DNA. Typically, multiple different loci are detected at the same time in a multiplex method and can be quantified as a result. This means that, in step (iv) of the method according to the invention, the nucleic acid amplified in step (iii) is preferably quantified via at least one known sequence region. In order to quantify the at least one known sequence region, it is possible to use, for example, a specific probe and/or sequence-specific primers. For the quantification, double-strand-specific fluorescent dyes and/or at least one specific probe can likewise be used.

The DNA can be quantified using a hybridization-mediated method or a sequencing method. Examples of the hybridization-mediated methods known to a person skilled in the art include quantitative polymerase chain reaction (PCR), real-time PCR, strand displacement amplification (SDA), transcription-mediated amplification (TMA), helicase-dependent amplification (HDA), recombinase polymerase amplification (RPA), loop-mediated isothermal amplification (LAMP), SMart amplification process (SMAP), or else microarray-based methods (e.g. Affymetrix, Illumina, Agilent). Microarray-based methods (microarray methods for short) are preferred hybridization-mediated methods in the context of the present invention. Microarray methods are understood to mean methods in which 10 or more nucleic acid sequences are detected in parallel on surfaces. Said surfaces generally bear nucleic acid sequences which are used for the detection of 10 or more nucleic acid sequences. The sequences immobilized on the surfaces do not necessarily have to be nucleic acids, but can for example also be modified nucleic acids or else PNAs, and other molecules are also possible. The surfaces used in microarray methods are in particular curved or planar surfaces of different materials. Examples of the DNA sequencing methods include Pyrosequencing (Biotage AB, 454 Life Sciences (Roche), Solexa® (Illumina® Inc.) or SOLiD Sequencing (Applied Biosystems). Further suitable quantification methods are known to a person skilled in the art.

Particular preference is given to the detection of one or more sequence segments of the amplified DNA being carried out by means of quantitative real-time PCR.

In addition, particular preference is given to the detection of one or more sequence segments of the amplified DNA being carried out by means of a microarray method. Likewise, particular preference is given to the detection of one or more sequence segments of the amplified DNA being carried out by means of a sequencing method.

In one particular embodiment of the method according to the invention, the quantity of one or more sequence segments of the DNA in the sample treated with the methylation-dependent nuclease is compared with the quantity of said sequence segment(s) of the DNA in a control sample which had not been treated with a methylation-dependent nuclease.

The quantity of particular sequence segments (loci) in a sample or on a DNA can be expressed, for example, as a threshold cycle (C_{T}) when the quantification is carried out by means of real-time PCR. The C_{T} value indicates in which PCR cycle the fluorescence values indicative of a particular sequence in each case are above the measurable threshold and is therefore a measure of how much DNA of the particular sequence was originally in the sample: a low C_{T} value indicates a relatively large original amount of the particular DNA sequence in the sample compared to a higher C_{T} value, since fewer amplification cycles were required in order to detect the said sequence in the sample. If the efficiency of a particular amplification system is known, it is possible to calculate back from a C_{T} value to the originally existing amount of the particular DNA sequence in the sample by means of a comparison with standard values. The C_{T} values after treatment of the sample with the methylation-dependent nuclease can be compared, for example, with corresponding values without treatment of the sample with the methylation-dependent nuclease. This can be done for instance by calculating the difference (“delta C_{T}”) of C_{T}^{untreated}−C_{T}^{treated}. The smaller this difference, the greater the distance of the corresponding DNA sequence from a methylation site. In other words: the closer a sequence segment is to a methylation site in the DNA which is cut by the methylation-dependent nuclease in the process according to the invention, the less efficiently said sequence segment is amplified. In extreme cases, amplification of the sequence segment in question is not possible at all, especially when said sequence segment itself was methylated and is therefore cut by the methylation-dependent nuclease.

To ensure the comparability of the amounts of particular DNA sequence segments from samples treated and untreated with the methylation-dependent nuclease, the conditions under which amplification and quantification or detection take place should in each case be virtually identical. This means that it is advantageous to also carry out in parallel the method according to the invention without the addition of the methylation-dependent to the sample, i.e. without step (ii).

As mentioned, the method according to the invention produces more amplicons from the central regions of a DNA fragment cut by means of the methylation-dependent nuclease than from the peripheral regions. The exact position of the methylated site(s), i.e. the cleavage sites, does not necessarily have to be known. When any desired sequence region is selected for analysis, a statement about the methylation can even be made if the sequence region analysed lies only in the proximity of the methylated site. Such an analysis cannot indicate how strongly a particular site is methylated. Thus, such an analysis cannot establish whether a sequence in the genome is methylated, for example, to a certain extent, but merely indicates whether there are in general methylated regions in the vicinity of the sequence analysed. Therefore, said analysis is used primarily to determine the global methylation pattern and not to quantify the degree of methylation of defined sequences. In particular embodiments of the method according to the invention, an analysis of the sequence representation can be carried out thus on the treatment of the DNA with the methylation-dependent nuclease and subsequent amplification.

The DNA polymerase which is used during a PCR or a quantitative (real-time) PCR (qRT-PCT) in the context of the method according to the invention is preferably a polymerase from a thermophilic organism or is a thermostable polymerase or is a polymerase selected from the group consisting of Thermus thermophilus (Tth) DNA polymerase, Thermus acquaticus (Taq) DNA polymerase, Thermotoga maritima (Tma) DNA polymerase, Thermococcus litoralis (Tli) DNA polymerase, Pyrococcus furiosus (Pfu) DNA polymerase, Pyrococcus woesei (Pwo) DNA polymerase, Pyrococcus kodakaraensis KOD DNA polymerase, Thermus filiformis (Tfi) DNA polymerase, Sulfolobus solfataricus Dpo4 DNA polymerase, Thermus pacificus (Tpac) DNA polymerase, Thermus eggertsonii (Teg) DNA polymerase, Thermus brockianus (Tbr) and Thermus flavus (Tfl) DNA polymerase.

In the qRT-PCR, fluorescently labelled primers and/or probes can be used, for example LightCycler probes (Roche), TaqMan probes (Roche), Molecular Beacons, Scorpion primers, Sunrise primers, LUX primers or Amplifluor primers. Probes and/or primers can contain, for example, covalently or non-covalently bonded fluorescent dyes, for example fluorescein isothiocyanate (FITC), 6-carboxyfluorescein (FAM), xanthene, rhodamine, 6-carboxy-2′,4′,7′,4,7-hexachlorofluorescein (HEX), 6-carboxy-4′,5′-dichloro-2′,7′-dimethoxyfluorescein (JOE), N,N,N′,N′-tetramethyl-6-carboxyrhodamine (TAMRA), 6-carboxy-X-rhodamine (ROX), 5-carboxyrhodamine-6G (R6G5), 6-carboxyrhodamine-6G (RG6), rhodamine 110; coumarins, such as umbelliferone, benzimides, such as Hoechst 33258; phenanthridines, such as Texas Red, ethidium bromide, acridine dyes, carbazole dyes, phenoxazine dyes, porphyrin dyes, polymethine dyes, cyanine dyes, such as Cy3, Cy5, Cy7, SYBR Green, BODIPY dyes, quinoline dyes and Alexa dyes.

A person skilled in the art is aware that, in qRT-PCR, double-strand-specific fluorescent dyes, for example ethidium bromide, SYBR Green, PicoGreen, RiboGreen etc., can also be used independently of primers and probes.

The appropriate conditions for a quantitative PCR are known to a person skilled in the art. This concerns, for example, the primer design, the choice of appropriate processing temperatures (denaturation, primer annealing, elongation), the number of PCR cycles, the buffer conditions.

In the context of the present invention, the DNA is in particular genomic DNA. Genomic DNA is understood to mean a deoxyribonucleic acid which can be obtained from organisms and is partly methylated. The methylation can affect different bases and different positions. The genomic DNA can have been obtained from organisms by, for example, lysis and/or purification.

The origin of the nucleic acid to be analysed can differ. The nucleic acid can have been isolated, for example, from one or more organisms selected from the group comprising viruses, phages, bacteria, eukaryotes, plants, fungi and animals (e.g. mammals, especially primates). The nucleic acid can also originate, for example, from cellular organelles. In addition, the nucleic acid to be analysed can be a constituent of samples. Such samples can likewise differ in origin. For instance, the method according to the invention also provides the analysis of nucleic acids which are present in samples from body fluids, environmental samples or foodstuff samples.

In the context of the present invention, organisms are understood to mean any form of organic shells which contain nucleic acids. Examples of these include viruses, phages, prokaryotic and eukaryotic cells, cell assemblages or entire organisms. Said organisms can be used alive or dead. Said organisms can be in solution, pelleted or else associated with or bound to solid phases. “Organisms” can also mean a plurality of the same kind of organism, a plurality of different kinds of organism or else just one organism.

As mentioned, in the method according to the invention, lysis of the organism, cell or tissue containing the nucleic acid may also be necessary before the amplification. In the context of the present invention, the term “lysis” is understood to mean a process which results in nucleic acids and/or proteins being released from a sample material into the surroundings. In this process, the structure of the sample material can be destroyed, for example the shell of the sample material can be dissolved. In the context of the present invention, the term “lysis” is also understood to mean that the nucleic acid can escape from the sample material through small openings, for example pores, etc., in the shell of the sample material without destroying the structure of the sample material. For example, pores can be generated by lysis reagents. Furthermore, in the context of the present invention, the term “lysis” is to be understood to mean that nucleic acids and/or proteins of the sample material which already appears structurally destroyed or has small openings can be flushed out through the use of an additive. The lysis generates a lysate. The lysate can contain sample material of different organisms or of an individual organism, of different cells or of an individual cell, or of different tissues or of an individual tissue.

Purification of DNA is understood to mean that the DNA is separated from other ambient substances. This means that, after purification of the DNA, the sample is less complex with respect to the contents thereof.

The present invention also provides a kit for selectively accumulating non-methylated sequence segments of genomic DNA, comprising
a DNA polymerase,
a methylation-dependent nuclease
optionally: a buffer for the amplification reaction (e.g. containing buffer substance, dNTPs and/or primers)
optionally: a buffer for the endonucleolytic cleavage of methylated sequence segments by the methylation-dependent nuclease.

In addition, the present invention also provides a kit for determining the global methylation pattern of a genomic DNA, comprising
a DNA polymerase,
a methylation-dependent nuclease
optionally: a buffer for the amplification reaction (e.g. containing buffer substance, dNTPs and/or primers)
optionally: a buffer for the endonucleolytic cleavage of methylated sequence segments by the methylation-dependent nuclease.

The DNA polymerase of the kits according to the invention is preferably a polymerase from a thermophilic organism or is a thermostable polymerase or is a polymerase selected from the group consisting of Thermus thermophilus (Tth) DNA polymerase, Thermus acquaticus (Taq) DNA polymerase, Thermotoga maritima (Tma) DNA polymerase, Thermococcus litoralis (Tli) DNA polymerase, Pyrococcus furiosus (Pfu) DNA polymerase, Pyrococcus woesei (Pwo) DNA polymerase, Pyrococcus kodakaraensis KOD DNA polymerase, Thermus filiformis (Tfi) DNA polymerase, Sulfolobus solfataricus Dpo4 DNA polymerase, Thermus pacificus (Tpac) DNA polymerase, Thermus eggertsonii (Teg) DNA polymerase, Thermus brockianus (Tbr) and Thermus flavus (Tfl) DNA polymerase.

The methylation-dependent nuclease of the kits according to the invention is preferably selected from the group consisting of McrBC, McrA, DpnI, BisI, BlsI, GlaI, GluI, MalI and PcsI. Preference is given to McrBC and McrA, and particular preference is given to McrBC.

The methods and kits according to the invention can, for example, be used for selectively preparing, i.e. selectively accumulating, non-methylated sequence segments of genomic DNA. They can also be used for analysing the global methylation pattern in genomic DNA.

### DESCRIPTION OF THE DRAWINGS

FIG. 1: Illustration of an exemplary embodiment of the method according to the invention: what is shown is a genomic DNA (“gDNA”) consisting of non-methylated and methylated genomic segments. The methylated sites are indicated by “m”. In a first step, nucleolytic cleavage of the gDNA takes place following recognition of the methylated sequence segments by the methylation-dependent nuclease McrBC. In a second step, the cut DNA is amplified. In this exemplary embodiment, whole genome amplification (WGA) is performed. The gathering of amplified DNA molecules is indicated (“WGA ampl. DNA”). Distinctly more amplicons of cut DNA fragment are produced from the central regions than from the peripheral regions.

### EXAMPLES

### Example 1

### Exemplary Embodiment of the Method

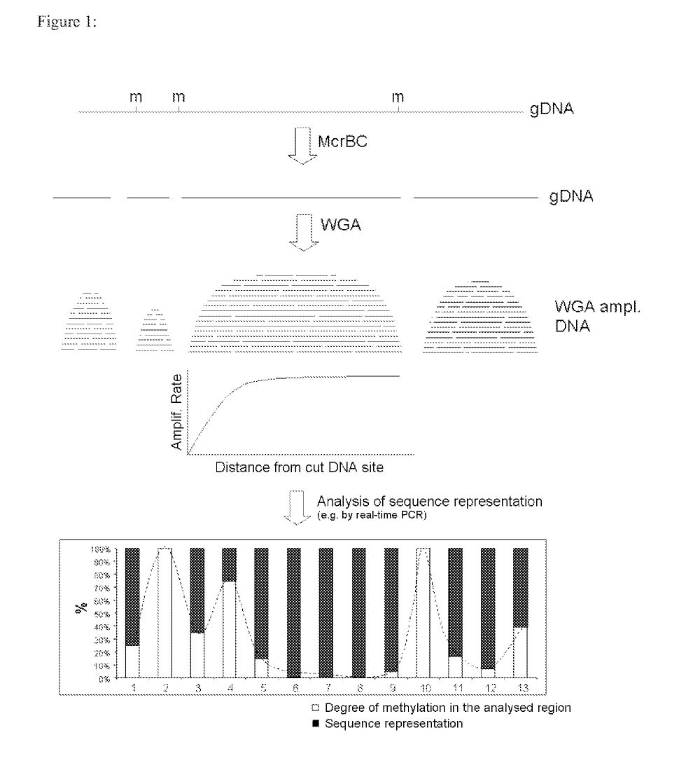

A genomic DNA (denoted by “gDNA”) consists of non-methylated and methylated genomic segments. The methylated sites are indicated by “m” in FIG. 1. In a first step, nucleolytic cleavage of the gDNA takes place following recognition of the methylated sequence segments by a methylation-dependent nuclease (indicated by “McrBC” in the FIGURE). In a second step, the cut DNA is amplified. In this exemplary embodiment, whole genome amplification is performed (indicated by “WGA” in the FIGURE). The gathering of amplified DNA molecules is indicated (“WGA ampl. DNA”). It can be clearly seen that more amplicons are produced from the central regions of a cut DNA fragment than from the peripheral regions. The result of this is the advantage that the exact position of the methylated site does not necessarily have to be known. When a region is selected for analysis, a statement about the methylation can even be made if the sequence region analysed lies only in the proximity of the methylated site. Such an analysis cannot indicate how strongly a particular site is methylated (i.e. it cannot indicate whether a sequence in the genome is methylated, for example, to an extent of 35%), but merely indicates whether there are in general methylated regions in the vicinity of the sequence analysed. Therefore, said analysis is preferably used to determine the global methylation pattern.

### Example 2

### Example of the Determination of the Methylation in Genomic Regions

Description of Experiment:

Genomic DNA was isolated from HepG2 cells using the QIAamp Kit (QIAGEN). 1 μg of the DNA was transferred to a reaction mix containing McrBC enzyme: said reaction mix (“+McrBC reaction mix”) contained 1 μg of DNA, 0.5 U/μl McrBC (NEB), 1×NEB2 buffer (NEB), 100 ng/μl BSA and 1 mM GTP. A further reaction mix (“−McrBC reaction mix”) contained the same components, but without McrBC enzyme. Both reaction mixes were incubated at 37° C. for 2 h followed by inactivation at 65° C. for 20 min. Subsequently, 10 ng were taken from the reaction mixes for a WGA reaction. The WGA reaction was performed using REPLI-g Midi reagents according to the REPLI-g Midi protocol for purified DNA. The WGA was carried out at 30° C. for 8 h followed by a 5 min inactivation at 65° C. To measure the sequence representation after WGA had taken place, a real-time PCR analysis was carried out. Here, three different genomic loci were analysed. The primers for the analysis are reported in table 1.

TABLE 1
Primers used for loci a, b and c from example 2

| | Primers |
|---|---|
| Locus a | TTC CCA CTC AAA ACT CCC AC |
| | ACA GGA ATG AGG GCA GCT AA |
| Locus b | TGCCCGCGTCCGTCCGTGAAA |
| | AGTCTCCGTCGCCGTCCTCGTC |
| Locus c | GGT AGG ATG ATT CTA GAA TGA CA |
| | GCC CAA ATT GGC TTC TTT TT |

For the real-time PCR, QuantiTect Sybr Green reagents (QIAGEN) and 10 ng of the respective WGA DNA were used in a 20 μl PCR reaction. The primer concentration in the analyses was 0.4 μM. The threshold cycles (C_{T} values) were recorded and are reported in table 2 below.

TABLE 2
Determined threshold values (C_{T} values)
for loci a, b and c from example 2

| | | Locus a | Locus b | Locus c |
|---|---|---|---|---|
| −McrBC | WGA 1 | 23.53 | 20.38 | 25.13 |
| | WGA 2 | 23.38 | 20.30 | 25.25 |
| +McrBC | WGA 3 | 31.35 | 28.04 | 25.22 |
| | WGA 4 | 30.66 | 27.61 | 24.13 |

When the mean of WGA 3 and 4 is subtracted from the mean of WGA 1 and 2, the sequence representation difference delta CT is obtained (table 3).

TABLE 3
Delta C_{T} values for loci a, b and c from example 2

| | Locus a | Locus b | Locus c |
|---|---|---|---|
| Delta C_{T} | 7.55 | 7.48 | −0.52 |

Result:

It can be seen in table 2 that the C₁ values (locus a and locus b) for WGA reactions 3 and 4 (“+McrBC reaction mixes”) are higher than for WGA reactions 1 and 2 (“− McrBC reaction mixes”). The higher C_{T} values (also evident through the delta C_{T} values) indicate a lower sequence representation. This means that locus a and locus b are present in a lower concentration in WGA 3 and 4 than in WGA 1 and 2. A lower concentration of locus a and locus b in WGA 3 and WGA 4 reveals that the McrBC enzyme has hydrolytically cut the DNA in the proximity of these loci. Since McrBC only cuts when the enzyme recognition sites in the DNA are methylated, it can be inferred that the DNA was methylated in the proximity of loci a and b.

The situation is different for locus c: the C_{T} values are comparable in WGA reactions 1-4. It can be inferred therefrom that no methylated sequences are to be found in the proximity of locus c.

### Example 3

### Example of the Determination of the Methylation in Genomic Regions in Different Genomic DNAs

Description of Experiment:

Genomic DNA was isolated from HepG2 cells and from the blood from four different test subjects (B1 to B4) using the QIAamp Kit (QIAGEN). 1 μg of the DNA was transferred to a reaction mix containing McrBC enzyme: said reaction mix (“+McrBC reaction mix”) contained 1 μg of DNA, 0.5 U/μl McrBC (NEB), 1×NEB2 buffer (NEB), 100 ng/μl BSA and 1 mM GTP. A further reaction mix (“−McrBC reaction mix”) contained the same components, but without McrBC enzyme. Both reaction mixes were incubated at 37° C. for 2 h followed by inactivation at 65° C. for 20 min. Subsequently, 10 ng were taken from the reaction mixes for a WGA reaction. The WGA reaction was performed using REPLI-g Midi reagents according to the REPLI-g Midi protocol for purified DNA. The WGA was carried out at 30° C. for 8 h followed by a 5 min inactivation at 65° C. To measure the sequence representation after WGA had taken place, a real-time PCR analysis was carried out. Here, three different genomic loci were analysed. The primers for the analysis are reported in table 4.

TABLE 4
Primers used for loci a to g from example 3

| | Primers (5′-3′ sequence) | Sequence ID |
|---|---|---|
| Locus a | TTCCCACTCAAAACTCCCAC | SEQ ID NO: 1 |
| | ACAGGAATGAGGGCAGCTAA | SEQ ID NO: 2 |
| Locus b | TGCCCGCGTCCGTCCGTGAAA | SEQ ID NO: 3 |
| | AGTCTCCGTCGCCGTCCTCGTC | SEQ ID NO: 4 |
| Locus c | GGTAGGATGATTCTAGAATGACA | SEQ ID NO: 5 |
| | GCCCAAATTGGCTTCTTTTT | SEQ ID NO: 6 |
| Locus d | GTCTTTAGCTGCTGAGGAAATG | SEQ ID NO: 7 |
| | AGCAGAATTCTGCACATGACG | SEQ ID NO: 8 |
| Locus e | CAACTGGCCCTGTCGTTCC | SEQ ID NO: 9 |
| | CCATGTTGCTGACCCGGTAG | SEQ ID NO: 10 |
| Locus f | ACTGGTTGGAGTTGTGGAGACG | SEQ ID NO: 11 |
| | TGGAATGCTTGAAGGCTGCTC | SEQ ID NO: 12 |
| Locus g | AACTGAATGGCAGTGAAAACA | SEQ ID NO: 13 |
| | CCCTAGCCTGTCATTGCTG | SEQ ID NO: 14 |

For the real-time PCR, QuantiTect Sybr Green reagents (QIAGEN) and 10 ng of the respective WGA DNA were used in a 20 μl PCR reaction. The primer concentration in the analyses was 0.4 μM. The threshold cycles (C_{T} values) were recorded and are reported in table 5 below.

When the C_{T} values obtained from the WGA reactions with prior McrBC treatment of the genomic DNA are subtracted from the C_{T} values obtained from the WGA reactions without prior McrBC treatment of the DNA, the delta C_{T} value is obtained. The delta C_{T} value is a measure of how strongly the representation of the loci examined differs between the +McrBC reaction mixes and the −McrBC reaction mixes. A very high delta C_{T} value indicates that the representation in the +McrBC reaction mixes has distinctly decreased with respect to the −McrBC reaction mixes.

TABLE 5
Delta C_{T} values for loci a to g from example 3

| | Locus a | Locus b | Locus d | Locus e | Locus f | Locus g |
|---|---|---|---|---|---|---|
| HepG2 | 6.6 | 7.8 | 4.6 | 6.2 | 7.7 | 18.0 |
| B 1 | 4.4 | 2.5 | 2.6 | 7.7 | 13.0 | 14.4 |
| B 2 | 3.6 | 3.4 | 2.8 | 7.9 | 10.8 | 10.2 |
| B 3 | 4.0 | 3.3 | 3.4 | 4.4 | 11.1 | 10.0 |
| B 4 | 5.3 | 2.7 | 3.9 | 9.7 | 9.4 | 13.5 |

Result:

From the table of the delta C_{T} values, it can be seen that the delta C_{T} values are similar when B1 to B4 are compared. For instance, the delta C_{T} values for locus b are between 2.5 and 3.5. In contrast, in the case of the genomic DNA from HepG2 cells, the delta C_{T} value of locus b is distinctly different from the delta C_{T} values of the blood from donors B1 to B4. This indicates that HepG2 cells have a different methylation pattern compared to the blood from test subjects B1 to B4. In the case of locus f, a lower delta C_{T} value is found in HepG2 cells than in blood, indicating stronger methylation at the site of or in the proximity of locus fin blood.

1. Method for selectively amplifying non-methylated sequences of a DNA comprising the steps of
(i) providing a sample comprising a DNA Which is methylated at least one site,
(ii) treating the DNA in the sample with a methylation dependent nuclease, and
(iii) random-primed sequence amplification of the DNA cut using the methylation-dependent nuclease.

2. Method according to claim 1, wherein the amplification is conducted isothermally.

3. Method according to claim 2, wherein the amplification is carried out by means of strand displacement amplification.

4. Method according to claim 2, wherein the amplification is carried out by means of random-primed PCR.

5. Method according to claim 1, comprising the additional step of
(iv) detecting at least one sequence segment of the amplified DNA.

6. Method according to claim 5, wherein the detection comprises the quantification of at least one sequence segment of the amplified DNA.

7. Method according to claim 6, wherein the detection of one or more sequence segments of the amplified DNA is carried out by means of a hybridization-mediated method.

8. Method according to claim 7, wherein the detection of one or more sequence segments of the amplified DNA is carried out by means of a quantitative real-time PCR.

9. Method according to claim 7, Wherein the detection of one or more sequence segments of the amplified DNA is carried out by means of a microarray-based method.

10. Method according to claim 6, wherein the detection of one or more sequence segments of the amplified DNA is carried out by means of a sequencing method.

11. Method according to claim 5, wherein the quantity of one or more sequence segments of the DNA in the sample treated with the methylation-dependent nuclease is compared with the quantity of said sequence segment(s) of the DNA in a control sample which had not been treated with a methylation-dependent nuclease.

12. Method according to claim 1, wherein the methylation-dependent nuclease is selected from the group consisting of McrBC, McrA, DpnI, BisI, BlsI, GlaI, GluI, MalI and PcsI.

13. Method according to claim 1, wherein steps (ii) and (iii) are carried out simultaneously.

14. Method according to claim 1, wherein the DNA is genomic DNA.

15. Kit for selectively accumulating non-methylated sequence segments of genomic DNA, comprising
a DNA polymerase,
a methylation dependent nuclease,
optionally: a buffer for the amplification reaction (e.g. containing buffer substance, dNTPs and/or primers), and
optionally: a buffer for the endonucleolytic cleavage of methylated sequence segments by the methylation-dependent nuclease.

16. Kit for determining the global methylation pattern of a genomic DNA, comprising
a DNA polymerase,
methylation-dependent nuclease,
optionally: a buffer for the amplification reaction (e.g. containing buffer substance, dNTPs and/or primers), and
optionally: a buffer for the endonucleolytic cleavage of methylated sequence segments by the methylation-dependent nuclease.

17. Use of the method according to claim 1 for selectively preparing (selectively accumulating) non-methylated sequence segments of genomic A.

18. Use of the method according to claim 1 for analysing the global methylation pattern in genomic DNA.

19. Use of the kit according to claim 15 for selectively preparing (selectively accumulating) non-methylated sequence segments of genomic DNA.

20. Use of the kit according to claim 16 for selectively preparing (selectively accumulating) non-methylated sequence segments of genomic DNA.
